(19) United States
(12) Patent Application Publication
(10) Pub. No.: US 2024/0034966 A1
(43) Pub. Date: Feb. 1, 2024

(54) A CELL CULTURE SYSTEM CONTROLLER

(71) Applicant: Verso Biosense Group Limited, Abingdon Oxfordshire (GB)

(72) Inventors: Joanna Smart, Abingdon, Oxfordshire (GB); Joseph Cefai, Abingdon, Oxfordshire (GB)

(21) Appl. No.: 18/265,855

(22) PCT Filed: Dec. 2, 2021
(86) PCT No.: PCT/GB2021/053147

§ 371 Date: Jun. 7, 2023

(30) Foreign Application Priority Data
Dec. 11, 2020 (GB) 2019628.3

(51) Int. Cl. C12M 1/107; C12M 1/00; C12M 1/34; A61B 5/00

(52) U.S. Cl. CPC C12M 21/04; C12M 41/14; C12M 41/26; C12M 41/34; A61B 5/4325; A61B 5/6875; A61B 5/0031

(57) ABSTRACT

A cell culture system controller for controlling the environment of a cell culture system, the controller configured to receive intrauterine data and adjust an environmental parameter of a cell culture system environment over a period of time based on the intrauterine data.

### FIELD OF THE INVENTION

The present invention relates to a cell culture system controller, a cell culture system comprising the cell culture system controller, and a method of controlling a cell culture system.

### BACKGROUND OF THE INVENTION

An incubator is a device used to develop and maintain cell cultures, such as human embryos. The environmental conditions within the incubator are maintained at constant levels in order to ensure productive conditions for culturing cells. Existing incubators have internal environments that can control the levels of temperature, oxygen content and pH in the incubation medium, and will actively monitor at least one of these parameters to maintain it at a constant level. Each clinic will typically follow a standardised protocol such that, whilst the conditions are maintained at constant levels, the exact conditions considered optimal for embryo incubation vary between clinics.

The incubation period is typically between 3 to 5 days, depending on the particular protocol followed by the clinic, at which point the embryo is implanted into a patient.

### SUMMARY OF THE INVENTION

A first aspect of the invention provides a cell culture system controller for controlling the environment of a cell culture system, the controller configured to receive intrauterine data and adjust one or more environmental parameters of a cell culture system environment over a period of time based on the intrauterine data.

A second aspect of the invention provides a method of controlling the environment of a cell culture system, the method comprising: receiving intrauterine data, and providing a cell culture system controller to adjust one or more environmental parameters of the cell culture system by using the intrauterine data.

By adjusting one or more environmental parameters based on the intrauterine data, the cell culture system can be adjusted to replicate the environment of a patient's uterus. This can increase the chances of an embryo being viable when it is implanted into the uterus. The environmental parameters can be tailored to a particular recipients intrauterine conditions, to the environmental parameters identified as favorable in a clinical trial or other study, or to patients with similar conditions (e.g. coeliac disease). This may be particularly beneficial to patients who have previously failed IVF implantation.

A further aspect of the invention provides a cell culture device, comprising the cell culture system controller of the first aspect.

The intrauterine data may comprise monitored uterine conditions.

The one or more environmental parameters may be adjusted to replicate the environment of a single patient's uterus.

The period of time may be at least 24 hours.

The controller of the first aspect may be configured to generate a time-dependent function based on the intrauterine data and wherein the one or more environmental parameters may be adjusted according to the time-dependent function.

The method of controlling the environment of a cell culture system of the second aspect may further comprise: generating a time-dependent function based on the intrauterine data; and, adjusting the one or more environmental parameters according to the time-dependent function.

The profile of the time-dependent function may be shaped to adjust the one or more environmental parameters to a maximum value and/or minimum value at least twice over the period of time.

The controller may be configured to use the intrauterine data to adjust the temperature linearly between a first value at a first time and a second value at a second time, preferably wherein the first and second times are at least 12 hours apart, and more preferably at least 24 hours apart.

The intrauterine data may comprise at least two measurements of an environmental parameter.

The one or more environmental parameters may be selected from: temperature, oxygen concentration, and pH.

The one or more environmental parameters may be selected from: pressure, light and humidity.

The one or more environmental parameters may be selected from: a nutrient, a hormone, and a carbon dioxide concentration.

The controller may be configured to receive intrauterine data and adjust two or more environmental parameters of the cell culture system environment over a period of time based on the intrauterine data, and preferably three or more environmental parameters.

The cell culture system may be configured to culture embryonic cells.

The cell culture device may further comprise an intrauterine device for placing into a uterus, the intrauterine device being configured to record the intrauterine data.

The cell culture device may further comprise a user accessible memory with a plurality of time-dependent profiles stored and be configured to pre-set an environmental parameter.

The cell culture device may further comprise a user interface which is connected to the user-accessible memory to select at least one time-dependent profile.

The user interface may be used to provide the intrauterine data to the cell culture system controller.

The controller may be coupled to an environmental element of the cell culture system, and the environmental element may be configured to adjust one or more of the environmental parameters of the cell culture system environment.

The environmental element may include a heating element, and the heating element may be configured to adjust the temperature of the cell culture system environment.

The environmental element may include a gas supply, and the gas supply may be configured to adjust the gas flow of the cell culture system environment.

The environmental element may include a chemical supply channel, and the chemical supply channel may be configured to adjust one or more of the pH level, nutrient level, and hormone level of the cell culture system environment.

The cell culture device may further comprise a storage device, wherein the intrauterine data is stored on the storage device.

The cell culture device may further comprise a receiver for receiving the intrauterine data wirelessly.

The method of controlling the environment of a cell culture system of the second aspect may include: wherein the controller adjusts the temperature of the cell culture system environment.

The method of controlling the environment of a cell culture system of the second aspect may include: adjusting the one or more environmental parameters to replicate the environment of a single patient's uterus.

### BRIEF DESCRIPTION OF THE DRAWINGS

Embodiments of the invention will now be described with reference to the accompanying drawings, in which:

FIG. 1 shows a cell culture system controlling an environmental parameter of an incubator according to a first example;

FIG. 2A shows a female patient;

FIG. 2B shows a uterus comprising an intrauterine device;

FIG. 3 shows a linear increase in an environmental parameter towards the end of an incubation cycle;

FIG. 4 shows a temperature profile configured to compensate for the removal of embryos during the incubation cycle;

FIG. 5 shows a set of data points of intrauterine data recorded over a period of time;

FIG. 6 shows a time-dependent function based on the intrauterine data shown in FIG. 5;

FIG. 7 shows a cell culture system including a user interface;

FIG. 8 shows the time-dependent profiles of three environmental parameters.

### DETAILED DESCRIPTION OF EMBODIMENT(S)

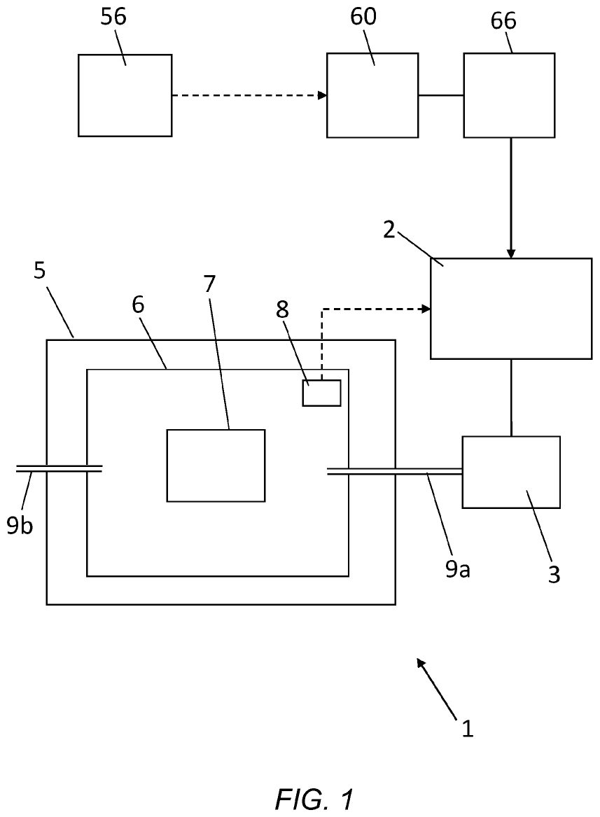

FIG. 1 shows a cell culture device controller 2. The cell culture system controller may be part of a cell culture device 1. The cell culture system controller 2 is arranged to control a cell culture system environment 6 over a period of time based on intrauterine data. The cell culture system environment 6 may be the environment of an incubator 5.

The environment 6 may be controlled by an environmental element 3. The incubator 5 may be suitable for incubating human embryos or animal embryos. Reference to embryo refers to an embryo at any suitable stage of development, for example the blastocyst stage or zygote stage.

In alternative examples, the incubator may be a cuvette, flushing medium system, or any other suitable device for providing an environment for cell culturing.

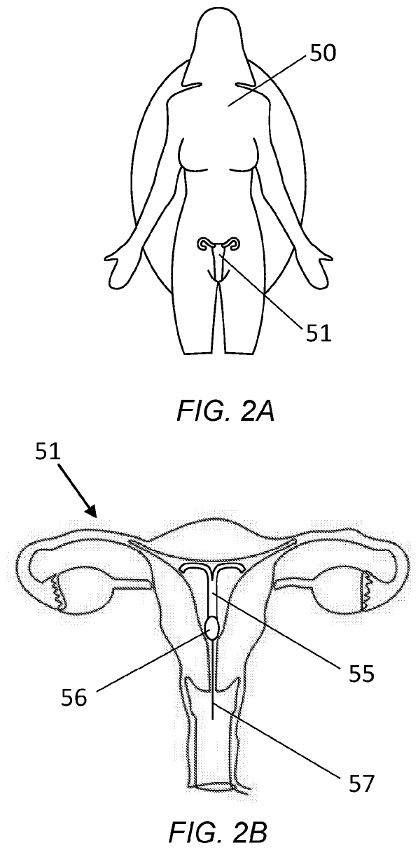

Embryos are typically stored in an incubator 5 for a period of time, such as 2 to 5 days, and then transferred to the uterus 51 of a female patient 50 (See FIG. 2A). The period of time may be at least 24 hours.

It has been found that the viability of human embryos in incubation may be increased by more closely replicating the conditions of the uterus 51, and in particular taking into account the variability in the environmental conditions of the uterus 51 over time. To do this, an intrauterine device (IUD) 55 may be placed into the uterus 51 to monitor the conditions of the uterus 51 over a time period, as shown in FIG. 2B. The collected data may be recorded by the intrauterine device and/or transmitted to a storage device. The data may be transmitted to a receiver wirelessly via a transmitter 56, as shown in FIG. 2B, or transmitted via a connecting wire such as IUD string 57.

The shape and positioning of the intrauterine device 55 in the uterus 51 may be similar to conventional intrauterine devices, such as T-shaped copper coils having a IUD string 57 extending from the device 55, as shown in FIG. 2B, although it will be clear that the intrauterine device 55 may be any shape suitable for collecting the necessary data from the uterus 55.

As shown in FIG. 1, the cell culture system environment 6 may be an incubator environment 6 of an incubator 5. An embryo dish 7, or similar device, may be arranged inside the incubator environment 6 in order to house an embryo. The incubator environment 6 may be monitored by one or more sensors 8 placed inside the incubator environment 6. The sensors 8 may be arranged to monitor one or more environmental parameters of the incubator environment 6, such as temperature, oxygen concentration, pH, pressure, light, humidity, nutrient concentration, hormone concentration, and carbon dioxide concentration.

The sensors 8 may provide environmental data from the cell culture system environment 6 to the cell culture system controller 2. The cell culture system controller 2 may process the data to determine an input rate of the one or more environmental elements 3. The input rate of the one or more environmental elements 3 may also be determined based on data received from the intrauterine device 55. The intrauterine data may be transmitted to the cell culture system controller 2 via a transmitter 56 and receiver 60 arrangement, as shown in FIG. 1. The data may be stored on a storage device 66.

The environment of the incubator 5 may be adjusted via one or more environmental elements 3. The cell culture system controller 2 may control the one or more environmental elements 3 so as to controllably vary one or more environmental parameters of the incubator 5. The cell culture device 1 may include an input line 9a connected between the cell culture system environment 6 and an environmental element 3 to facilitate inflow of one or more environmental parameters. The cell culture device 1 may include an output line 9b arranged to allow the outflow of one or more environmental parameters. The rate of outflow through the output line 9b may be passively controlled, or may be controlled by the cell culture system controller 2.

In some examples, the input and output lines 9a, 9b may be a common line. In some examples, an input line 9a and an output line 9b may be provided for inflow and outflow of each environmental parameter. Each environmental element 3 may be controlled by a common cell culture system controller 2, or may be controlled by separate cell culture system controllers 2.

The incubator 5 of the cell culture system 1 may include a plurality of cell culture system environments 6, with each environment 6 being sealed from the other respective environments 6. Each cell culture system environment 6 may be controlled by separate environmental elements 3, or an environmental element 3 may control two or more of the cell culture system environments 6.

The environmental element 3 may be any device suitable for adjusting an environmental parameter of the incubator 5, such as a heating element, gas supply, or chemical supply. In one example, the controller 2 controls the operation of a heating element 3 such that the temperature of the cell culture system environment is adjusted to a prescribed value.

As previously discussed, the environmental element 3 may be a heating element suitable for adjusting the temperature of the environment of the incubator 5.

Incubation temperatures are typically determined based on the particular protocol of a clinic, such that the temperature considered optimal for incubation is held at a fixed temperature for all embryos at a particular clinic. For example, the incubation temperature may be 36.2 degrees C.

As a result, the end temperature experienced by the embryo is typically different to the temperature experienced by the embryo when it is implanted into the patient. This temperature difference will typically be different for each patient, but the resulting change in temperature can damage the embryo or otherwise shock the embryo, such that the chances of survival are decreased.

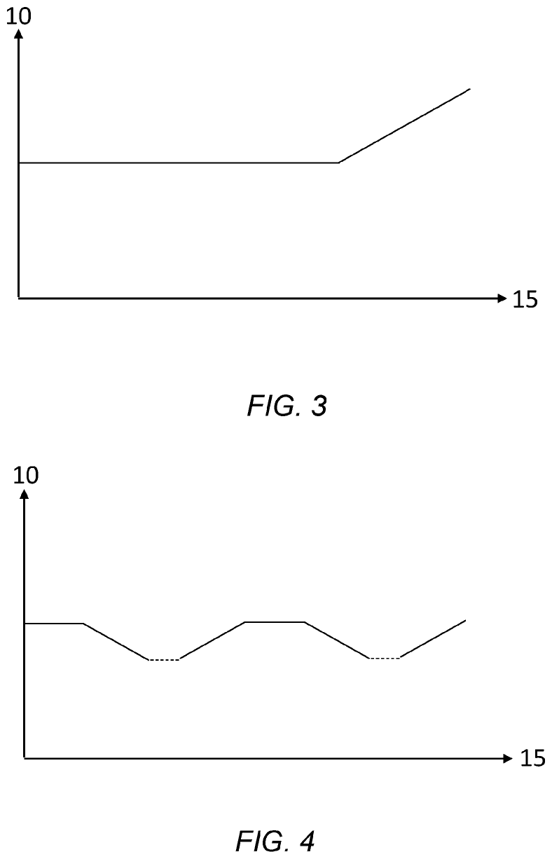

FIG. 3 shows the variation of an environmental parameter (y-axis) 10 of an environment of the incubator 5 against time (x-axis) 15. In this case the environmental parameter 10 is temperature. It has been found that the chances of survival for the embryo may be increased by increasing the temperature in the incubation environment in the final stages over an incubation period, such as the final 12 hours or 24 hours of incubation.

In this example, the incubation temperature is initially fixed at a first temperature for a first period of time, for example 72 hours, and then the temperature is linearly increased, for example for a second period of 12 hours, to a second temperature that takes into account the temperature of the patient's uterus. The second temperature may be based on the temperature of a patient's uterus, or a temperature similar to the patient's uterus. The second period may be any suitable period, for example 24 hours.

Whilst the example in FIG. 3 shows a linear increase in temperature, the second temperature may alternatively be decreased with respect to the first temperature. The increase or decrease may be a linear function, or may follow any other non-linear function, such as a parabolic function, and/or vary in stages with periods of constant temperature between. It will also be understood that in additional examples, any environmental parameters of the incubator may be varied in the same way, either individually or collectively at the same time. For example, the oxygen concentration, carbon dioxide concentration, pH, pressure, light, humidity, iron concentration, nutrient concentration, and hormone concentration may all be varied according to a specified function in the final stages of incubation.

FIG. 4 shows a function according to an alternative example. In-situ monitoring of embryos allows the development of the embryos to be monitored inside the cell culture system environment 6 without disturbance. In-situ monitoring is not always possible, and so the embryos may need to be periodically removed from the incubator 5. For example, the embryo may be inspected once per day.

When each embryo is removed from the incubation environment to check its development, the temperature of the embryo and the temperature of the cell culture system environment 6 (and other environmental parameters) may be affected. This sudden change in environmental conditions may damage the embryos being removed, as well as damage those embryos left inside the cell culture system environment 6.

To account for this, the environmental parameters may be varied over a period of time prior to the inspection of an embryo, such that the effects of opening the incubator to remove the embryo are reduced. The environmental parameters may similarly be varied over a second period of time after the embryos are inspected and the incubator subsequently closed.

In one example, the incubator temperature may be nominally set at 36.2 degrees Celsius. When inspection of the embryos is required, a protocol may be enacted such that the temperature is gradually reduced to a temperature closer to room temperature—such as 25 degrees C. When inspection is concluded, the embryo may be returned to the incubator 5 and then subsequently the temperature of the cell culture system environment 6 is increased back to 36.2 degrees Celsius.

As a result, the embryos may be acclimatised in order to avoid thermal shock.

It will be understood that any suitable environmental parameter, or combination of environmental parameters, may be adjusted based on an external environment in order to avoid shock to the embryo.

FIG. 4 shows the environmental parameter increasing and decreasing at equivalent linear rates. In other examples, the environmental parameter may be adjusted at any suitable rate. The function may be linear or non-linear.

The environmental parameter may be adjusted to a first value to prepare the embryo for extraction from the incubator 5, and adjusted to a second value prior to reintroduction of the embryo into the incubator 5. The first and second values may be the same. The first and second values may be different.

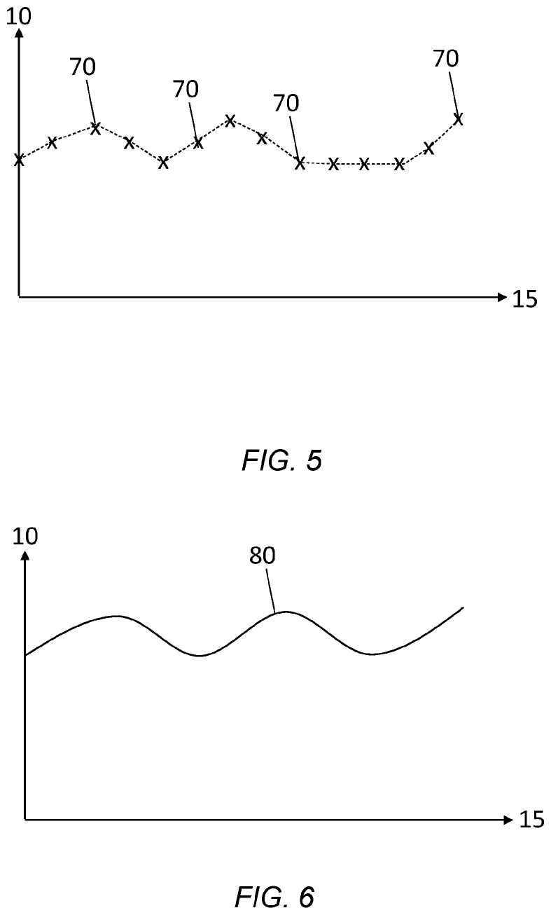

FIG. 5 shows a set of data points 70 of intrauterine data recorded by the intrauterine device 55 over a period of time (note only some data points are labelled in FIG. 5 to simplify the figure). The intrauterine data 70 may be stored on a storage device or user accessible memory 66. The intrauterine data may be representative of any suitable environmental parameter, for example: oxygen concentration, carbon dioxide concentration, pH, pressure, light, humidity, nutrient concentration, iron concentration, and hormone concentration may all be varied according to the given function in the final stages of incubation.

The intrauterine data points 70 may be used directly to determine the variation of one or more environmental parameters. For example, the cell culture system environment 6 may be varied according to the values prescribed by the data points 70, with any variation between adjacent data points 70 being calculated by interpolation between those adjacent points 70.

At least two intrauterine data points 70 may be provided to define a variation of the one or more environmental parameters, although the required data points 70 will depend on the amount of variation of the environmental parameters of a time, with the accuracy of the device 1 being improved by the use of additional data points. Preferably at least ten intrauterine data points 70 are provided.

The intrauterine data may be used to create a time-dependent function 80, as shown in FIG. 6. The function 80 may approximate the intrauterine data 70, thereby smoothing any noise in the intrauterine data and minimising the effects of any data errors. The function 80 may be selected from a list of predetermined functions, or a bespoke function may be fitted to the intrauterine data points 80. The predetermined functions may be stored in a storage device or user accessible memory 66.

The time-dependent function 80 may be selected based on one or more intrauterine data points 70. For example, a single measurement may be taken from a patient and used to select from a list of predetermined function profiles. Two or more measurements may be taken from a patient at different times, and either used to select from a list of predetermined function profiles or used to develop a best-fit function.

The profile of the time-dependent function 80 may be shaped to adjust the environmental parameters to a maximum value and/or a minimum value at least twice over the period of time. The period of time may be 12 hours. The period of time may be 24 hours. The period of time may be between 3 and 5 days.

The controlled variation of the environmental parameters, i.e. the time-dependent profile of the environmental parameters, may have a plurality of local maxima and minima. A plurality of time-dependent profiles may be selected over the incubation period of an embryo. For example, a different time-dependent profile may be selected for each 24 hour period of incubation.

The function 80 may be a function of any suitable order (e.g. first order, second order, third order) required to provide the optimal conditions for an embryo.

The cell culture device 1 may be a fully automated system that measures parameters of an individual patient and automatically sets the incubator 5 for the embryos. Alternatively or in addition, there may be some medical clinician intervention at points during the incubation cycle. The intervention may be prior to incubation of the embryo. The intervention may be during incubation of the embryo. The intervention may comprise checking the data and/or function. The intervention may comprise altering the data and/or function.

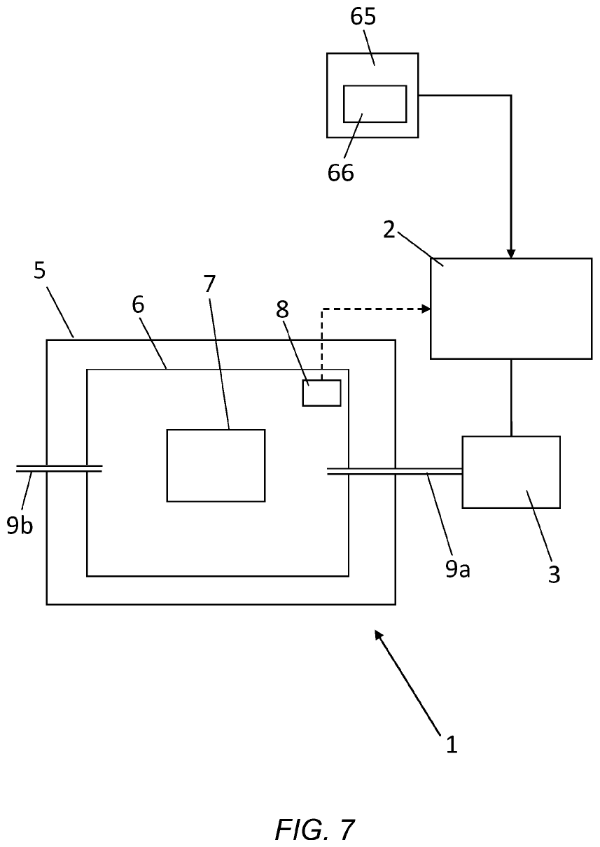

The cell culture device 1 may include a user interface 65, as shown in FIG. 7. The user interface 65 may be connected to a user-accessible memory 66. The user accessible memory 66 may store a plurality of time-dependent profiles that can be selected by the cell system controller 2 to control one or more environmental parameters. The time-dependent profile may be selected by a user via the user interface 65, such that a clinician can choose which profile to use according to the personal requirements of the patient.

The user interface 65 may be used to enter intrauterine data. The data may be entered manually by a clinician. For example, a maximum and minimum temperature of the time-dependent profile may be selected.

The environmental parameters may be controlled based on the intrauterine data of a single patient and used to control the environmental parameters of an cell culture system environment 6 incubating an embryo for that particular patient. The environmental parameters may be controlled based on intrauterine data from a different patient to the intended recipient of the embryo.

The environmental parameters may be controlled based on intrauterine data averaged from multiple patients. For example, the data may be averaged from a population of patients that have shown successful implantation of an embryo, or from a population of patients in a similar demographic. A time-dependent profile for controlling the environmental parameters may be selected based on this averaged data.

The cell culture system controller 2 may control two or more environmental parameters. The controller 2 may receive intrauterine data related to the two or more environmental parameters and control the cell culture system environment over a period of time based on the intrauterine data.

The cell culture system controller 2 may control three environmental parameters. The cell culture system controller 2 may be coupled to three environmental elements 3 each configured to adjust one of the three environmental parameters of the cell culture system environment. In one example, a first environmental element 3 may include a heating element configured to adjust the temperature of the cell culture system environment, a second environmental element 3 may include a gas supply configured to adjust the gas flow of the cell culture system environment, and a third environmental element 3 may include a chemical supply channel is configured to adjust the pH level of the cell culture system environment.

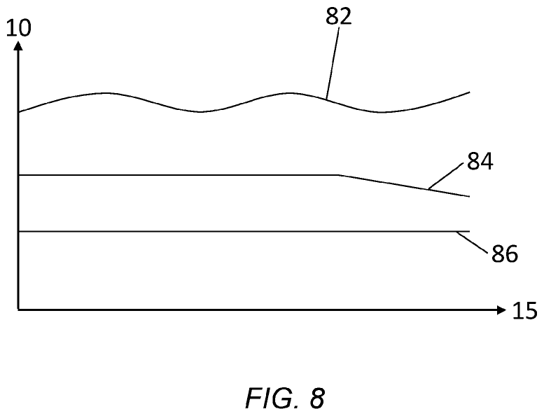

The environmental elements 3 controlling each of the three environmental parameters may adjust the environmental parameters based on different intrauterine data and/or different time-dependent functions 82, 84, 86, for example as shown in FIG. 8.

A first environmental parameter may be adjusted according to a first time-dependent function 82. The first time-dependent function 82 may be a sine function. For example, the oxygen concentration may be adjusted according to a sine function such that the oxygen concentration is adjusted between a maximum and a minimum on a daily (24 hour) cycle during the incubation period.

A second environmental parameter may be adjusted according to a second time-dependent function 84. The second time-dependent function 82 may comprise a substantially constant portion over a first time period, and a linearly decreasing portion over a second time period after the first time period. For example, the temperature may be held at approximately 36.2 degrees Celsius during a first time period, and then linearly decreased to a lower temperature towards the end of the incubation period to avoid thermal shock to the embryo when exposed to the external temperature outside the incubator 5.

A third environmental parameter may be adjusted according to a third time-dependent function 84. The third time-dependent function 82 may be held at a substantially constant value. For example, the pH may be held at substantially 7.3 pH throughout the incubation period. Alternatively, the environmental parameter may be controlled so that it is maintained between upper and lower limits, for example the pH may be maintained between 7.2 and 7.4 but allowed to freely vary within these limits.

Where the word ‘or’ appears this is to be construed to mean ‘and/or’ such that items referred to are not necessarily mutually exclusive and may be used in any appropriate combination.

Although the invention has been described above with reference to one or more preferred embodiments, it will be appreciated that various changes or modifications may be made without departing from the scope of the invention as defined in the appended claims.

1. A cell culture system controller for controlling the environment of a cell culture system, the controller configured to receive intrauterine data and adjust one or more environmental parameters of a cell culture system environment over a period of time based on the intrauterine data.

2. The cell culture system controller of claim 1, wherein the intrauterine data comprises monitored uterine conditions.

3. The cell culture system controller of claim 1, wherein the one or more environmental parameters are adjusted to replicate the environment of a single patient's uterus.

4. The cell culture system controller of claim 1, wherein the period of time is at least 24 hours.

5. The cell culture system controller of claim 1, wherein the controller is configured to generate a time-dependent function based on the intrauterine data and wherein the one or more environmental parameters are adjusted according to the time-dependent function.

6. The cell culture system controller of claim 5, wherein the profile of the time-dependent function is shaped to adjust the one or more environmental parameters to a maximum value and/or minimum value at least twice over the period of time.

7. The cell culture system controller of claim 1, wherein the controller is configured to use the intrauterine data to adjust the temperature linearly between a first value at a first time and a second value at a second time, preferably wherein the first and second times are at least 12 hours apart, and more preferably at least 24 hours apart.

8. The cell culture system controller of claim 1, wherein the intrauterine data comprises at least two measurements of an environmental parameter.

9. The cell culture system controller of claim 1, wherein the one or more environmental parameters are selected from: temperature, oxygen concentration, pH, pressure, light, humidity, a nutrient, a hormone, and a carbon dioxide concentration.

10. (canceled)

11. (canceled)

12. The cell culture system controller of claim 1, wherein the controller is configured to receive intrauterine data and adjust two or more environmental parameters of the cell culture system environment over a period of time based on the intrauterine data, and preferably three or more environmental parameters.

13. The cell culture system controller of claim 1, wherein the cell culture system is configured to culture embryonic cells.

14. A cell culture device, comprising the cell culture system controller of claim 1.

15. The cell culture device of claim 14, further comprising an intrauterine device for placing into a uterus, the intrauterine device configured to record the intrauterine data.

16. The cell culture device of claim 14, wherein the cell culture device further comprises a user accessible memory with a plurality of time-dependent profiles stored and is configured to pre-set an environmental parameter.

17. The cell culture device of claim 16, further comprising a user interface which is connected to the user-accessible memory to select at least one time-dependent profile.

18. The cell culture device of claim 17, wherein the user interface is used to provide the intrauterine data to the cell culture system controller.

19. The cell culture device of claim 14, wherein the controller is coupled to an environmental element of the cell culture system, and the environmental element is configured to adjust one or more of the environmental parameters of the cell culture system environment.

20. The cell culture device of claim 19, wherein the environmental element includes one or more of a heating element configured to adjust the temperature of the cell culture system environment, a gas supply configured to adjust the gas flow of the cell culture system environment, and a chemical supply channel configured to adjust one or more of the pH level, nutrient level, and hormone level of the cell culture system environment.

21. (canceled)

22. (canceled)

23. The cell culture device of claim 14, further comprising a storage device, wherein the intrauterine data is stored on the storage device.

24. The cell culture device of claim 14, further comprising a receiver for receiving the intrauterine data wirelessly.

25. A method of controlling the environment of a cell culture system, the method comprising:
receiving intrauterine data, and
providing a cell culture system controller to adjust one or more environmental parameters of the cell culture system by using the intrauterine data.

26. The method of controlling the environment of a cell culture system of claim 25, wherein the intrauterine data comprises monitored uterine conditions.

27. The method of controlling the environment of a cell culture system of claim 25, further comprising adjusting the one or more environmental parameters to replicate the environment of a single patient's uterus.

28. The method of controlling the environment of a cell culture system of claim 25, further comprising:
generating a time-dependent function based on the intrauterine data; and, adjusting the one or more environmental parameters according to the time-dependent function.

29. The method of controlling the environment of a cell culture system of claim 25, wherein the controller adjusts the temperature of the cell culture system environment.
